(19) United States
(12) Patent Application Publication
(10) Pub. No.: US 2009/0233284 A1
(43) Pub. Date: Sep. 17, 2009

(54) METHOD FOR THE SIMULTANEOUS DETECTION OF MUTATIONS OCCURRING IN RELATED GENOMES, EXPLOITING MOBILITY VARIATIONS IN 2-D ELECTROPHORESIS OF DNA DUPLEXES UNDERGOING DIFFERENTIAL HELIX-COIL TRANSITION IN A DENATURING GRADIENT

(75) Inventors: Vittorio Sgaramella, Lodi (IT); Simona Panelli, Lodi (IT); Giuseppe Damiani, Pavia (IT)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(73) Assignee: PARCO TECNOLOGICO PADANO SRL, Lodi (IT)

(21) Appl. No.: 12/162,345

(22) PCT Filed: Jan. 27, 2006
(86) PCT No.: PCT/IB2006/000266

§ 371 Date: Nov. 25, 2008

(51) Int. Cl. C12Q 1/68

(52) U.S. Cl. 435/6

(57) ABSTRACT

A method for the simultaneous mutation detection exploiting the variable electrophoretic mobility of related DNA samples, caused by the differential onset of helix-coil transition in denaturing gradients, comprises the following subsequent steps: a) preparation of DNA samples; b) fragmentation of the samples through a first restriction; c) terminal labeling of the fragments of each genome with a different fluorochrome; d) mixing the samples; e) carrying out a simultaneous second restriction, converting each long duplex into a set of shorter fragments with two of them labeled at only one of their ends e) carrying out a 2D separation of the mixed digestion products in order to obtain: a first dimension consisting of a standard 20 polyacrylamide gel; and a second dimension against a denaturing gradient; f) spotting, picking of singly labeled spots and sequencing of their DNA.

### TECHNICAL FIELD OF THE INVENTION

The present invention relates to a method for detecting any DNA mutations, even if rare and unpredicted, arising in two or more related genomes, otherwise identical.

As examples of such mutations many situations can be referred to, in which genomic rearrangements play pivotal roles, either during normal development and growth or in relation to pathological conditions. Among them consider the following:

(a) bacteria becoming resistant to antibiotics;

(b) bacteria changing from avirulence to virulence;

(c) somatic cells extracted from either different tissues or organs of the same organism;

(d) somatic cells extracted from either healthy or pathological portions of the same tissue/organ, etc.

The method based on the present invention may be applied to the simultaneous analysis of two or more different genomes, depending on the availability of different labelling markers: examples are two or more tissue-specific genomes of the same individual, or two or more anthropomorphic primates.

Accordingly, a further possibility arises, namely the comparison of genomes that are largely different but may exhibit limited homologies, i.e. islands of identical sequences embedded in regions of different composition: examples are any two unrelated organisms inhabiting the same habitat, be it a living system or an inorganic niche.

The present invention may be applied to any system for separating mixtures of fragments of two or more genomes, after the genomes have been restricted and labelled by means of marker compounds which do not affect the chemical, electrophoretic and physical properties of the resulting fragments, but produce different and distinguishable signal patterns.

The invention may also be applied to the analysis of the cellular transcriptomes, after conversion of mRNA into cDNA, as detailed later.

### BACKGROUND ART

Genetics is the science that studies mutations, i. e. changes in the genome of an organism as they are formed either spontaneously or following appropriate mutagenic treatments. Traditionally mutations are detected through a phenotypic analysis of mutants, i. e. through the study of their differences in features and/or properties if compared to a “wild type” organism.

With the advent of molecular genetics, mutations can be characterized through a genotypic analysis, i. e., through the resolution of the DNA sequence of those particular genes reputed to be responsible of the mutated trait(s).

However, several mutations do not bring about phenotypic changes, or if they do so, they may be difficult to predict as to the resulting phenotype.

In more recent times, especially after the diffusion of large scale-sequencing, it has become possible to carry on what has been called “reverse genetics”, an approach exploiting the massive and indiscriminate sequencing of genomes. This leads to the identification of many mutations, disregarding of their effects on the phenotype of the organisms.

In spite of the ever increasing efficiency of sequencing, especially following the recent completion of the Human Genome Project (International Human Genome Sequencing Consortium, Nature 431: 931, 2004), in order to detect the sites and the nature of mutations it remains unpractical to sequence the whole genomes of the mutated organisms. This is especially true if their sizes are in the range of the billions bp, as in the cases of the genomes of higher organisms, animals as well as plants.

Several methods have been proposed for using and detecting markers, analysing transcriptome patterns, and performing whole genome and whole transcriptome amplifications.

For example, Document WO-A-2005/081867 discloses a method to detect a biomarker in saliva for the diagnosis of oral cavity cancer or infections, and for a more general monitoring of human health. Biomarkers may be extracellular mRNAs, and are indicators of specific biological properties that can be used to measure either the progress of several diseases or the effects of any treatment. The method comprises a transcriptome analysis of saliva, i.e. the detection of a transcriptome pattern in the cell-free saliva. Furthermore, said document describes a method to detect genetic alterations by analyzing saliva.

Document US-A-2004/0209298 discloses several methods and compositions for whole genome and transcriptome amplifications. In a particular aspect there is a method of amplifying a genome comprising library generation followed by its amplification. In specific embodiments the library generation step utilizes specific primer mixtures and a DNA polymerase, wherein the specific primer mixtures are designed to eliminate the ability to self-hybridize and/or hybridize to other primers, and to efficiently and frequently prime the replication of nucleic acid templates.

For these analyses it would be highly desirable to control a technology that enables the researcher to spot any differences between two or more closely related genomes, such as those descending from a common ancestor. This is even more desirable if the researcher has a minimal or no a priori knowledge of the eventual differences.

Such a technology is expected to be considerably useful in the light of recent data relative to unexpected alterations. These may consist in variations of chromosomal DNA sequences (changes in the succession of four standard bases, generally irreversible in the nuclear genomes, Muotri A. R., et al., Somatic mosaicism in neuronal precursor cells mediated by L1 retrotransposition. Nature 435: 903-910, 2005), or in epigenetic features (post-synthetic changes, mostly in C, and in chromatin components other than DNA, both of them irreversible, Kimmins S. and Sassone-Corsi P., Chromatin remodelling and epigenetic features of germ cells. Nature 434: 583-588, 2005).

All these phenomena are involved in many steps of the physiopathological development of complex organisms, and thus in their overall functionality, as well as in the evolution of the relevant species.

In order to appreciate minimal mutations such as single base substitutions, and deletions or insertions, even without any cue as to their nature, position, and effects, it would be greatly helpful to exploit physico-chemical properties which may be affected by such minimal variations, short of either analysing long DNA sequences or interpreting the eventual phenotype.

### ILLUSTRATION OF DRAWINGS

FIG. 1 shows a simplified pattern in which a fictitious analysis of a mixture of two DNA has been represented, each DNA being composed of two fragments, one being common to the two samples and the other being different.

### DESCRIPTION OF THE INVENTION

The present invention aims to fulfill the requirements described above, and to overcome the difficulties of the background art. The aim is thus to provide a method that allows the simultaneous mutation detection in two or more genomes, exploiting the variable 2D electrophoretic mobility of DNA fragments caused both by the different length and the differential onset of helix-coil transition in a denaturing gradient.

This is obtained by carrying out a method having the features disclosed in claim 1. The dependent claims outline advantageous ways of carrying out a said method.

Now, in order to appreciate minimal/rare mutations such as substitutions, deletions or insertions, even limited to single bases, the properties that may be affected by them should be exploited in the most direct and informative way.

According to the invention, the onset of helix/coil transition is exploited, during standard electrophoretic separation in a denaturing gradient. The helix-coil transition leads eventually to the complete denaturation of duplex DNA and involves a series of steps. When the DNA fragments migrate in a field of increasing denaturing power, be it thermal or chemical or both, they are exposed to increasing denaturing conditions. AS they begin the helix/coil transition, they reduce their electrophoretic mobility until they come essentially to a stop. This prevents the completion of their helix-coil transition and allows their subsequent recovery as double stranded DNA and their further analysis.

If the separation is performed in a polyacrylamide gel, fragments of about 100-1000 bp may be profitably handled. If under study are genomes sized as the humans', then the fragments number would fall between 1 to 10 million.

Such a large number of fragments would pose very serious difficulties. Thus a preliminary reduction of their overall number to a manageable amount is recommendable.

According to the present invention, this can be achieved by first cleaving the genomes with a rare cutting restriction endonuclease, with a target of at least eight bp (NotI, SacII, SfiI, etc). This treatment would cut the human genome in some fifty thousand fragments, still a large but a manageable number, but their size would be of the order of 50-100 kb.

The previous labelling of the ends (each sample with a different fluorochrome) and the subsequent restriction with a frequent cutting restriction endonuclease (HaeIII, DpnI, AluI, etc.) would bring the size of the fragments to the desired range (1 kb) with an increase in the number of those carrying the label limited to a doubling of the original rarely-cut terminally labelled fragments.

According to an advantageous form of embodiment of the present invention, samples are mixed and run simultaneously in a 2D electrophoretic system. A first dimension is exploited, where the electrophoretic mobility is based only on fragment size, whereas the second dimension employs a denaturing gradient, as indicated above. Therefore, according to the invention, the 2D-gel electrophoretic system may display several tens of thousands spots in a standard matrix-format, and provide an informative pattern. Gels similar in size to those used for the early phases of the Genome Project (up to 60×40 cm), can be envisaged for the display of such large number of spots.

### Description of a Form of Embodiment of the Invention

In order to carry out the method according to the present invention, DNA samples to be analysed and compared should first be suitably prepared.

For the preparation of the DNA samples to be analysed and compared, traditional extraction methods can be used (Sambrook J. and Russell D. W. Molecular Cloning: A laboratory manual, 3^{rd} edition. Cold Spring Harbor Laboratory Press, Cold Spring Harbor, N.Y. 1996). In the case of exceptionally scarce samples, an isothermal amplification procedure can be used: a recent version has been recently described by Hutchison et al. (C. H. Hutchison, H. O. Smith, C. Pfannkoch, J. C. Venter. Cell-free cloning using F29 DNA polymerase. Proc. Nat. Acad. Sci. USA, 102, 17332-17336, 2005). An improved procedure named LIMDA has been described (Ligase-Improved Multiple Displacement Amplification, Panelli S. et al. Ligation overcomes terminal underrepresentation in multiple displacement amplification of linear DNA. BioTechniques, 39: 174, 2005) and this procedure is the object of international patent application No. PCT/IB2005/001048. According to this procedure, the MDA of linear DNA is expanded to the following steps:

a) production of short, linear DNA templates;

b) ligation of said templates;

c) multiple displacement amplification of the ligated templates.

For the subsequent fragmentation of DNA, rare cutter restriction endonucleases such as NotI, SfiI etc may be used, as outlined above. They cleave a genome like ours into some 50-100 thousand fragments (Takahashi K. et al. DNA Insight: a web-based image processing system for large scale RLGS analysis. Genome Informatics 12: 212-221, 2001; Costello J. F. et al. Aberrant CpG-island methylation has non-random and tumour-type-specific patterns. Nat Genet. 24:132-8, 2000).

The serial use of different rare cutters (with targets rich in either AT or GC) allows the required systematic scanning of the different genomes under study.

At this stage each DNA is terminally labelled with a different fluorochrome (e. g., Cy2, Cy3, Cy5, etc.). After labelling, samples are mixed and hence on they are processed together. This improvement has the advantage of minimizing the introduction in the ensuing analysis of artefactual indications of diversity/identity between fragments. The mixture is subsequently exposed to a frequent cutter restriction endonuclease (DpnI, AluI, HaeIII, MspI, etc.). This digestion converts each long (rarely cut) duplex into a set of shorter (frequently cut) fragments, as such easier to manage. Of these, only the two corresponding to the original termini will carry the label and thus show up in the final pattern.

The mixed digestion products are then subjected to a 2D separation, possibly achieved by electrophoresis. The 1st dimension is a standard polyacrylamide gel. The 2nd dimension is run also in this matrix against a gradient of denaturing conditions increasing in parallel with the migration.

As the duplexes migrate on, they will eventually encounter a threshold of denaturing conditions such that they can initiate their helix/coil transition. Its onset slows down the migration of a duplex, and prevents its progressive entering into more denaturing regions of the gradient, thus avoiding undergoing complete denaturation. As a consequence duplexes of equal size but differing even in a point mutation should stop at different denaturant concentrations. The matrix is eventually scanned on an instrument like the Amersham Typhoon.

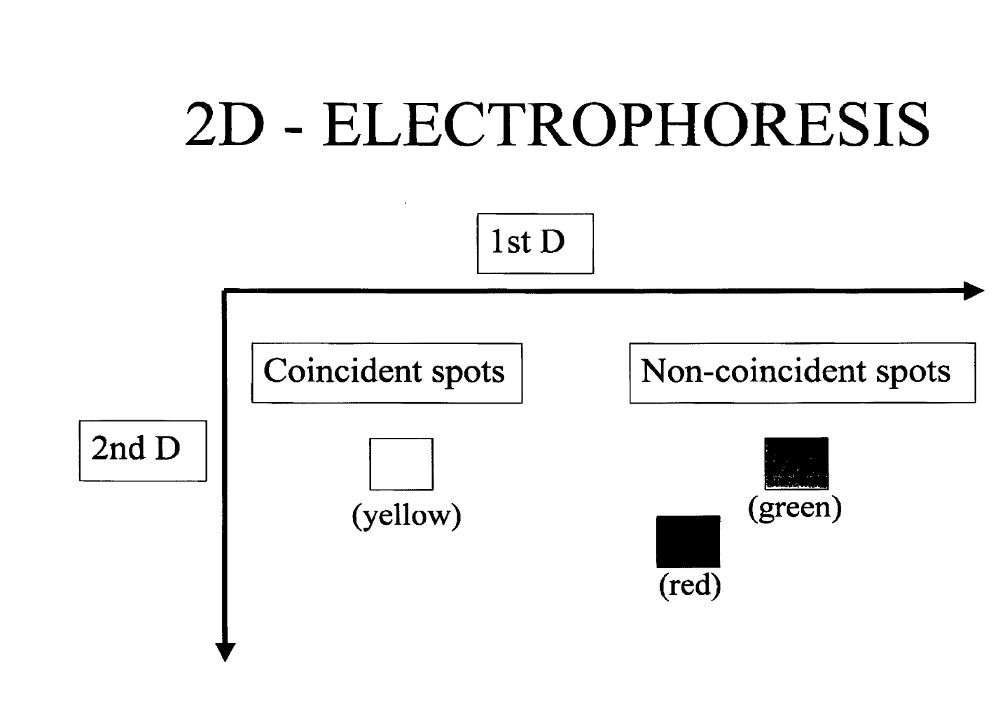

FIG. 1 shows a simplified pattern in which we have represented a fictitious analysis of a mixture of two DNA, each composed of two fragments, one being common to the two samples and the other being different. If the first DNA is labelled with a green fluorochrome, and the second with a red one, the final pattern will show a spot where the two identical fragments travel together (coincident spots), and give a hybrid signal, in this case yellow, and two different spots, labelled with the original fluorochromes, representing the two different fragments (non-coincident spots). These can be therefore identified and characterized by standard sequencing methods.

In the case of closely related genomes, the whole 2D matrix should be mostly occupied by comigrating spots containing duplexes of identical sequences, and characterized by hybrid fluorescence. Some spots of unique colour appear scattered on the matrix: they correspond to sequences present in only one of the genomes under examination. Their recovery as duplexes is possible with this technique, and allows their further molecular characterization.

According to the invention, an important application of the procedure involves the use of rare cutter restriction endonucleases that are inactive on target sequences that harbour methylated C (such as NotI). Since methylated C can be seen essentially as a fifth base in DNA, especially as it concerns the transcription of genes and in general the interaction of DNA with regulatory as well as structural elements (RNA, proteins), the presence or absence of methyl group on C may alter the function of genes and thus the physio-pathological properties of relevant cells (Robertson, K. D. DNA methylation and disease. Nature Reviews Genet. 5: 597-610, 2005). The different methylation status originates changes in the restriction patterns of the DNA under analysis, as already exploited by published techniques for the analysis of tumour cells (Rush, L. J., and Plass, C. Restriction landmark genomic scanning for DNA methylation in cancer: past, present, and future applications. Analytical Biochemistry 307: 191-201, 2002).

Particularly relevant to this point is the field of epigenetics (Bradbury J. Human epigenome project-up and running. PloS Biol. 1: 316-319, 2003). For what it concerns DNA, it is essentially dominated by transactions exploiting methylation/demethylation of genomic DNA (Solter D. Mammalian cloning: advances and limitations. Nat. Rev. Genet. 1: 199-207, 2000; Reik W. et al. Epigenetic reprogramming in mammalian development. Science 293:10898-193, 2001; Fairburn H. R. et al. Epigenetic reprogramming : how now, cloned cow? Current Biology 12: R68-R70, 2002). It is now well established that stem cell-based protocols, as well as cloning both for reproduction and regeneration, are heavily affected by epigenetic phenomena.

Another important form of embodiment of the method according to the present invention is related to the analysis of transcriptomes.

With transcriptomes the protocol partially exploits some previous observations (Asakawa, J. -I., et al. Two-dimensional cDNA electrophoresis revealing up-regulated human epididymal protein-1 and down-regulated CL-100 in thyroid papillary carcinoma. Endocrinology 143: 4422-4428, 2002) and accordingly starts with the isolation of messenger RNA (mRNA) from different sets of cells as previously outlined for the case of genomes.

The relevant steps are here carried out as follows:
1. synthesis of first-strand cDNA using an anchored oligo(d)T primer carrying at its 5′ end a recognition sequence for a rare cutter enzyme;
2. synthesis of double-stranded cDNA, blunt-ending, oligomerization-circularization, and LIMDA treatment as previously described;
3. restriction with the rare cutter whose recognition sequence has been introduced before, and fill-in of the ends with Cy2, Cy3, Cy5, as with genomic DNA;
4. restriction with a medium cutter;
5. mixing and electrophoretic analysis, carried out in a similar way as in the genomic analysis.

A particular care is due to the quantitative evaluation of the signals, since transcriptomes often differ in the relative abundances of their component mRNA, hence of their cDNA.

The possible applications of the method according to the present invention are numerous and interesting.

In animals, for example, putative stem cell characterization may be indicated, as well as analysis of cells to be used as nucleus donor for reproductive or therapeutic cloning, the detection of important genetic traits (biosynthetic capacity, resistance to parasites and pathogens), description of transgenic and genetically modified organisms, deciphering of sequences related to development and differentiation along pathways either physiological (aging) or pathological (neoplastic), speciation and evolutionary relationships, etc (Kazazian H. H. et al., LINE drive: retrotransposition and genome instability. Cell 110: 277-280, 2002).

In plants the analysis may be addressed to problems similar in variety and depth to those of animals: interest is high as it concerns the peculiar ability of plants to allow reproductive cloning, as opposed to the serious problems encountered in animals. In addition plant-specific problems can be listed: grafting affinity, sexual incompatibility, interspecific crossability, varietal fingerprinting, plant perennialism, apomixis. Finally the availability of secular plants represents an attractive system for the study of intraorganismal genomic differences (L. Bortolotti. Gli alberi monumentali d'Itala. Abete, Roma, RML 0044216).

In prokaryotes, particularly promising are those studies addressed to unravel the acquisition of virulence and of resistance to antibiotics. In general the analysis of genomic variations as due to the transposition of mobile elements appears as particularly rewarding (Howell-Adams B., and Seifert H. S. Molecular models accounting for the gene conversion reactions mediating gonococcal pilin antigenic variation. Mol. Microbiol. 37, 1146-1158, 2000). In the study of viruses, the method according to the present invention may be of great advantage in the mass decoding proposed for flu and other epidemic viral infections.

1. A method for the simultaneous mutation detection exploiting the variable electrophoretic mobility of related DNA samples, caused by the differential onset of helix-coil transition in denaturing gradients, comprising the following subsequent steps:
a) preparation of DNA samples;
b) fragmentation of the samples through a first restriction;
c) terminal labeling of the fragments of each genome with a different fluorochrome;
d) mixing the samples;
e) carrying Out a simultaneous second restriction, converting each long duplex into a set of shorter fragments with two of them labeled at only one of their ends;
e) carrying out a 2D separation of the mixed digestion products in order to obtain:
a first dimension consisting of a standard polyacrylamide gel; and
a second dimension against a denaturing gradient;
f) spotting, picking of singly labeled spots and sequencing of their DNA.

2. A method according to claim 1, wherein the DNA samples are prepared through traditional methods.

3. A method according to claim 1, wherein the DNA samples are prepared through a Ligase Mediated Multiple Displacement Amplification (LIMDA, see Panelli et al. BioTechniques, 2005) comprising the following subsequent steps:
a′) providing short, linear DNA templates with joinable ends;
b′) ligation of said templates;
c′) multiple displacement amplification of the ligated templates.

4. A method according to claim 1, wherein said fragmentation of samples is carried out with rare cutter restriction endonucleases.

5. A method according to claim 1, wherein the DNA fragments are labeled with different fluorescent markers.

6. A method according to claim 1, wherein the second restriction is carried out with frequent cutters.

7. A method according to claim 1, wherein the separation is carried out through 2D electrophoresis.

8. A method according to claim 7, wherein the denaturing gradient is of chemical nature, such as an increasing concentration of urea-formamide.

9. A method according to claim 7, wherein the denaturing gradient is of physical nature, such as an increasing temperature.

10. A method according to claim 1, carried out on transcriptomes, whereby messenger RNA is firstly isolated from different cells, including the following steps:
synthesis of cDNA, as from standard protocols;
endowing double-stranded cDNA of joinable ends, so that the cDNA can be oligomerised-circularised;
carrying out a Ligase Mediated Multiple Displacement Amplification;
restriction with medium cutters, fill-in of ends separately with Cy2, Cy3, Cy5, as with genomic DNA;
mixing, restriction with frequent cutter and analysis.
